SEQUENCE LISTING (12 sequences)

SEQ ID NO: 1 - protein, 121 aa, Mus musculus
ELKLEESGGGLVQPGGSMKLSCAASGFTFSDAWMDWVRQSPEKGLEWIAEIRSKANNYATYFAESVKGRFTISRDDSKSGVYLQMNNLRAEDTGIYFCTKLSLRYWFFDVWGAGTTVTVSS

SEQ ID NO: 2 - protein, 112 aa, Homo sapiens
EVQLQESGGGLVQPGGSLRLSCAASGFTFSDHYMDWVRQAPGKGLEWVGRTRNKATSYTTEYAASVKGRFTISRDDSKNSLYLQMNSLKTEDTGVYYCTKRWGQGTLVTVSS

SEQ ID NO: 3 - protein, 121 aa, Artificial Sequence
  Synthetic Polypeptide
EVQLQESGGGLVQPGGSLRLSCAASGFTFSDAWMDWVRQAPGKGLEWVGEIRSKANNYATYFAESVKGRFTISRDDSKNSLYLQMNSLKTEDTGVYYCTKLSLRYWFFDVWGQGTLVTVSS

SEQ ID NO: 4 - protein, 121 aa, Artificial Sequence; identical to SEQ ID NO: 3
  Synthetic Polypeptide

SEQ ID NO: 5 - protein, 113 aa, Mus musculus
DFVMTQTPLTLSVTIGQPASISCKSSQSLLDSDGKTYLNWLLQRPGQSPKHLIYLVSKLDSGVPDRFTGSGSGTDFTLRISRVEAEDLGVYYCWQSTHFPWTFGGGTKLEIKR

SEQ ID NO: 6 - protein, 113 aa, Homo sapiens
DIQMTQSPLSLSVTPGQPASISCKSSQSLLHSDGKTYLYWYQQKPGQSPQLLIYEVSNRFSGVPDRFSGSGSGTDFTLKISRVEAEDVGVYYCMQGIQLPWTFGQGTKVEIKR

SEQ ID NO: 7 - protein, 113 aa, Artificial Sequence
  Synthetic Polypeptide
DIQMTQSPLSLSVTPGQPASISCKSSQSLLDSDGKTYLNWYQQKPGQSPQLLIYLVSKLDSGVPDRFSGSGSGTDFTLKISRVEAEDVGVYYCWQSTHFPWTFGQGTKVEIKR

SEQ ID NO: 8 - protein, 113 aa, Artificial Sequence
  Synthetic Polypeptide
DFVMTQSPLSLSVTPGQPASISCKSSQSLLDYDGKTYLNWLQQKPGQSPKHLIYQVSKLNSGVPDRFSGSGSGTDFTLKISRVEAEDVGVYYCWQSTHFPWTFGQGTKVEIKR

SEQ ID NO: 9 - protein, 451 aa, Artificial sequence
  Synthetic polypeptide
EVQLQESGGGLVQPGGSLRLSCAASGFTFSDAWMDWVRQAPGKGLEWVGEIRSKANNYATYFAESVKGRFTISRDDSKNSLYLQMNSLKTEDTGVYYCTKLSLRYWFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial Sequence
  Synthetic Polypeptide
DFVMTQSPLSLSVTPGQPASISCKSSQSLLDYDGKTYLNWLQQKPGQSPKHLIYQVSKLNSGVPDRFSGSGSGTDFTLKISRVEAEDVGVYYCWQSTHFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1353 bp, Artificial sequence
  Synthetic polynucleotide
gaagtgcagcttcaggagtctggaggaggcttggtgcagcctggaggatccctgcgcctctcttgtgctgcctctggattcacttttagtgacgcctggatggactgggtccgccaggctccaggcaaggggcttgagtgggttggtgaaattagaagcaaagctaataattatgcaacatactttgctgagtctgtgaaagggaggttcaccatctcaagagatgattccaaaaatagtctgtacctgcaaatgaacagcttaaaaactgaggacactggcgtgtattactgtaccaagttatcactacgttactggttcttcgatgtctggggccaagggaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - DNA, 657 bp, Artificial sequence
  Synthetic polynucleotide
gatttcgtcatgacccagagcccactctctctgtccgtcacccctggacagccagcctccatctcttgcaagtcaagtcagagcctcttggattacgatggaaagacatatttgaattggttgcagcagaagccaggccagtctccaaaacatctcatctatcaggtgtctaaactgaactctggagtccctgacaggttcagtggcagtggatcagggacagatttcacactgaaaatcagccgggtggaggctgaggatgtgggggtttattactgctggcaaagtacacattttccgtggacgttcggccaggggaccaaggtggaaatcaaacggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2014/0314741 A1
(43) Pub. Date: Oct. 23, 2014

(54) Human Antibody against Interleukin-20 and Treatment for Inflammatory Diseases

(71) Applicant: DEVELOPMEN CENTER FOR BIOTECHNOLOGY, NEW TAIPEI CITY (TW)

(72) Inventors: CHIA-CHENG WU, NEW TAIPEI CITY (TW); CHAO-YANG HUANG, NEW TAIPEI CITY (TW); YU-YING LIN, NEW TAIPEI CITY (TW); YU-JUNG CHEN, NEW TAIPEI CITY (TW); JIANN-SHIUN LAI, NEW TAIPEI CITY (TW)

(73) Assignee: DEVELOPMEN CENTER FOR BIOTECHNOLOGY, NEW TAIPEI CITY (TW)

(21) Appl. No.: 13/865,671

(22) Filed: Apr. 18, 2013

(51) Int. Cl. C07K 16/24

(52) U.S. Cl. CPC C07K 16/244
USPC 424/133.1; 530/387.3

(57) ABSTRACT

FLB5M5 is a humanized monoclonal antibody with three mutated amino acids in the CDRs relative to its parental mouse anti-IL-20 monoclonal antibody 7E and five mutated amino acids of the light-chain framework region relative to the amino acids of the light-chain framework region of human Vκ2. FLB5M5 not only retains binding specificity toward IL-20 but also has a better binding affinity than 7E for IL-20. FLB5M5 is also less immunogenic than 7E to the human host in clinical application. A mutation in the light chain CDR to tyrosine increases binding affinity to IL-20. A method for treating rheumatoid arthritis using FLB5M5 is also disclosed.

### SEQUENCE LISTING

The Sequence Listing associated with this application is filed in electronic format via EFS-Web and hereby incorporated by reference into the specification in its entirety. The name of the text file containing the Sequence Listing is 1535600010sequence. The size of the text file is 17,864 bytes, and the text file was created on Apr. 18, 2013.

### BACKGROUND OF THE INVENTION

1. Field of the Invention

The field of the present invention is humanized antibodies against interleukin-20.

2. Background

Interleukin-20 (hereinafter “IL-20”) belongs to the IL-10 family and plays an important role in skin inflammation and various inflammation diseases. Previous studies demonstrated that IL-20 is a pleiotropic cytokine with chemoattractive, angiogenic, and osteoclastogenesis characteristics, as was evidenced by interfering with the binding of IL-20 to its receptors or blocking its signaling pathways. Inflammation and angiogenesis are essential for the pathogenesis of several inflammatory diseases such as psoriasis, atherosclerosis, stroke, and rheumatoid arthritis. Therefore IL-20 provides a therapeutic option for IL-20 induced inflammatory diseases and osteoporosis.

Rheumatoid arthritis, a chronic inflammatory disorder that principally affects synovial joints, is characterized by penetration of neutrophils, phagocytes, and lymphocytes into synovial membranes, which leads to excessive inflammation in joints. Cytokines and chemokines are known to play key roles in the development and progression of this disease.

### SUMMARY OF THE INVENTION

The present invention is directed toward FLB5M5, a humanized monoclonal antibody with three mutated amino acids in the complementarity determining regions (hereinafter “CDRs”), relative to its parental mouse anti-IL-20 monoclonal antibody 7E, and five mutated amino acids of the light-chain framework region, relative to the amino acids of the light-chain framework region of human Vκ2. A method for treating rheumatoid arthritis using FLB5M5 is also disclosed.

One aspect of this invention relates to FLB5M5. FLB5M5 not only retains binding specificity toward IL-20 but also has a better binding affinity than 7E for IL-20. FLB5M5 is also less immunogenic than 7E to the human host in clinical application.

Another separate aspect of this invention is the inclusion of a tyrosine amino acid in CDR1 of the light chain variable domain.

Another separate aspect of the invention is the combination of the tyrosine in CDR1 of the light-chain variable domain, along with the mutation of other amino acids in the CDRs.

Another separate aspect of the invention relates to a method of producing FLB5M5 by sequencing mouse monoclonal antibody 7E, selecting a framework donor antibody, replacing CDRs of the framework donor antibody with CDRs from the mouse monoclonal antibody 7E, replacing five amino acids from the framework donor antibody light-chain framework region with amino acids from the mouse monoclonal antibody 7E, and mutating three amino acids from the CDRs.

Yet another separate aspect of the present invention relates to a method of treating Rheumatoid Arthritis using FLB5M5 by administering FLB5M5 in an amount sufficient to treat the disease.

### BRIEF DESCRIPTION OF THE DRAWINGS

FIG. 1 provides a comparison of the amino acids of 7E (SEQ ID NO: 1), human homolog (SEQ ID NO: 2), HH12 (SEQ ID NO: 3), and FLB5M5 (SEQ ID NO: 4), in the V_{H} region.

FIG. 2 provides a comparison of the amino acids of 7E (SEQ ID NO: 5), human homolog (SEQ ID NO: 6), HH12 (SEQ ID NO: 7), and FLB5M5 (SEQ ID NO: 8), in the V_{L} region.

FIG. 3 is a graphical representation of the results from a proliferation assay.

FIG. 4 provides the AS profiles of healthy, diseased, and treated rats.

FIG. 5 provides the sequence of the heavy chain of a humanized antibody, FLB5M5 (SEQ ID NO: 9).

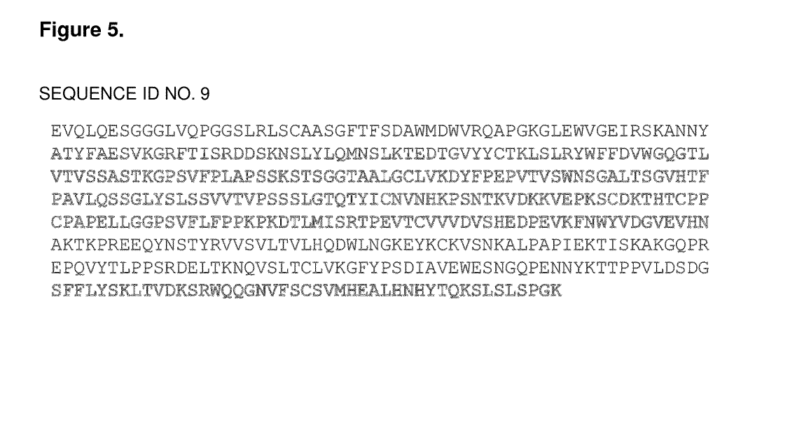

FIG. 6 provides the sequence of the light chain of a humanized antibody, FLB5M5 (SEQ ID NO: 10).

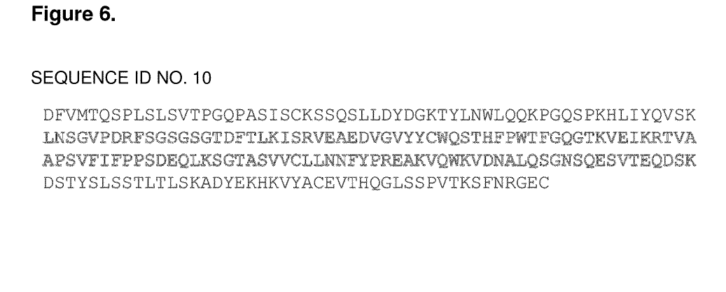

FIG. 7 provides a polynucleotide sequence (SEQ ID NO: 11) encoding the heavy chain of the humanized antibody, FLB5M5.

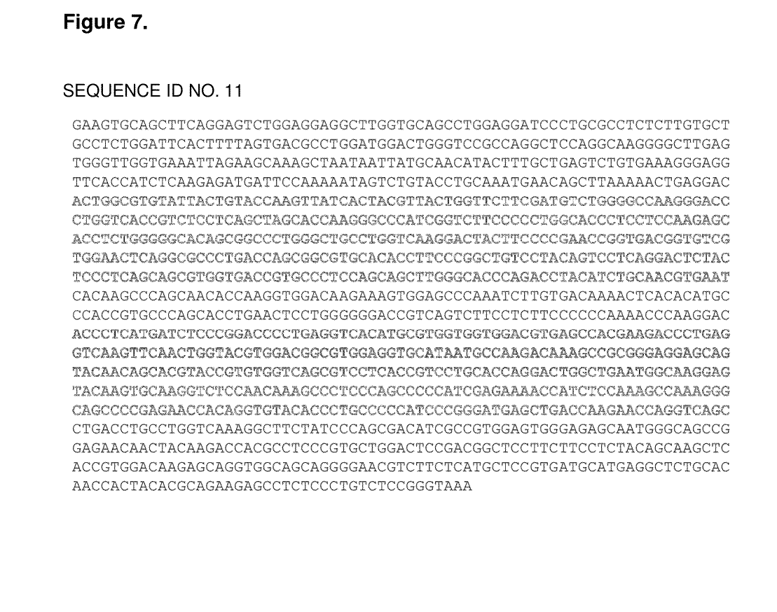

FIG. 8 provides a polynucleotide sequence (SEQ ID NO: 12) encoding the light chain of the humanized antibody, FLB5M5.

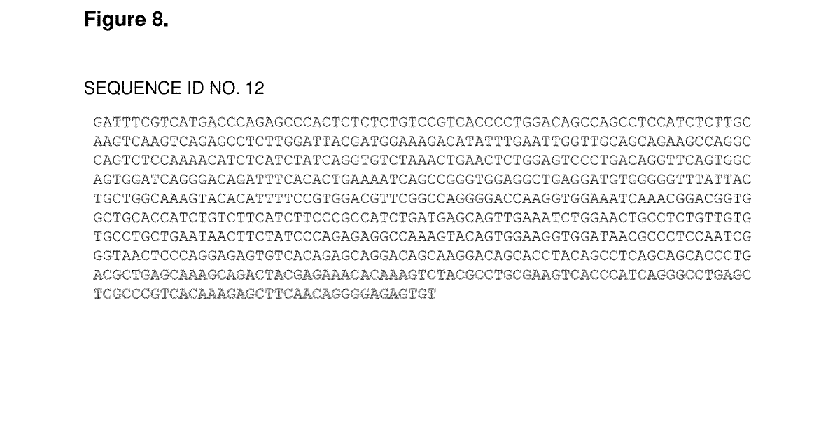

### DETAILED DESCRIPTION OF THE INVENTION

We discovered that a humanized monoclonal antibody with three mutated amino acids in the CDRs, relative to its parental mouse anti-IL-20 monoclonal antibody 7E, and five mutated amino acids of the light-chain framework region, relative to the amino acids of the light-chain framework region of human Vκ2, surprisingly not only retains binding specificity to IL-20, but also has a better binding affinity than the parental mouse anti-IL-20 monoclonal antibody 7E. We also surprisingly found that the humanized monoclonal antibody is less immunogenic to the human host, during clinical application, than the parental mouse anti-IL-20 monoclonal antibody 7E.

We also discovered that a tyrosine residue located in the light-chain variable domain CDR1 plays a role in increasing the binding affinity for the humanized anti-IL-20 antibodies. In addition, we have shown that the combination of this tyrosine with other amino acid residues mutation can further enhance the binding affinity.

A list of abbreviations employed herein is as follows:
Ab: antibody
BaF3: murine precursor B cells
CDRs: complementarity determining regions
C_{H}: heavy-chain constant domain
C_{L}: light-chain constant domain
HEPES: 4-(2-hydroxyethyl)-1-piperazineethanesulfonic acid
IL-20: interleukin-20
LTM: look-through mutagenesis
PBS: phosphate buffered saline
PCR: polymerase chain reaction
PTH: hind paw thickness
AS: arthritis score
RPMI medium: Roswell Park Memorial Institute medium
scFv: single-chain variable fragment
SD rats: Sprague-Dawley rats
V_{L}: light-chain variable domain
V_{H}: heavy-chain variable domain

The present disclosure is directed toward a humanized antibody with a tyrosine residue at position 27E, which is in V_{L} CDR-1. The present disclosure is also directed toward a humanized antibody, FLB5M5 (heavy chain: SEQUENCE ID NOS. 9 and 11; light chain: SEQUENCE ID NOS. 10 and 12), derived from mouse monoclonal antibody 7E. The present invention is also directed toward a process for the production of the humanized antibody by: (1) sequencing mouse monoclonal antibody 7E; (2) selecting a framework donor antibody; (3) replacing CDRs of the framework donor antibody with CDRs from the mouse monoclonal antibody 7E; (4) replacing five amino acids from the framework donor antibody light-chain framework region with amino acids from the mouse monoclonal antibody 7E; and (5) mutating three amino acids from the CDRs.

As used herein, the term “antibody” includes the whole antibody as well as fragments of the whole antibody with a binding affinity for IL-20 similar to that of the whole antibody.

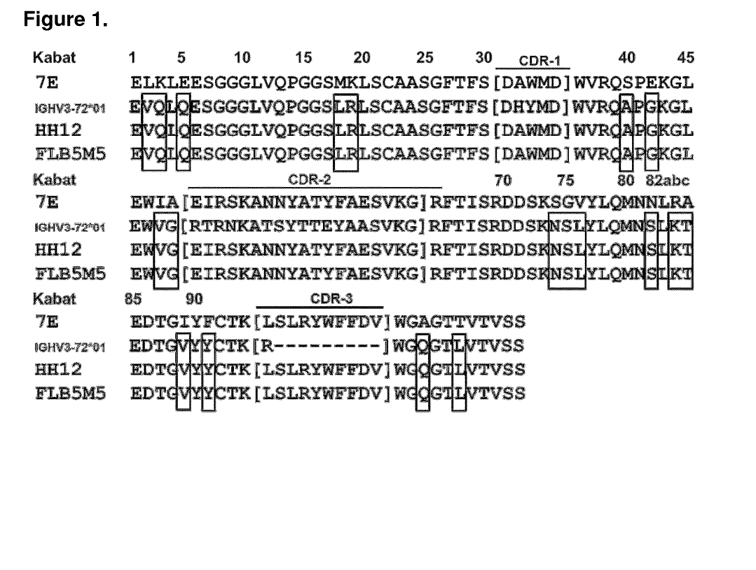

In preparing the humanized antibody, the amino acid sequence of the mouse monoclonal antibody 7E was determined through methods known in the art. That sequence was then used as input to search for human germ-line V_{L} and V_{H} sequences with the highest degree of homology with the 7E framework regions. The human homolog to 7E was, thus, determined to be IGHV3-72*01 for the V_{H} and IGKV2D-29*02 for V_{L}. The CDRs of the human homolog were replaced with the CDRs of 7E by methods known in the art, to give the humanized anti-IL-20 antibody HH12. FIG. 1 provides a comparison of the amino acids of 7E, human homolog, HH12, and FLB5M5, in the V_{H} region. In FIG. 1, amino acids that differ between FLB5M5 and HH12 in the V_{H} region are enclosed in rectangles.

Once HH12 was prepared, it was further modified to optimize the binding affinity for IL-20 and to minimize its immunogenic effect on the human host, giving FLB5M5. To do so, five amino acids from the HH12 light-chain framework region were replaced with amino acids from the mouse monoclonal antibody 7E, and three amino acids from the CDRs were mutated. These modifications can be performed in either order, i.e. modifying the framework first and then the CDRs or modifying the CDRs first and then the framework.

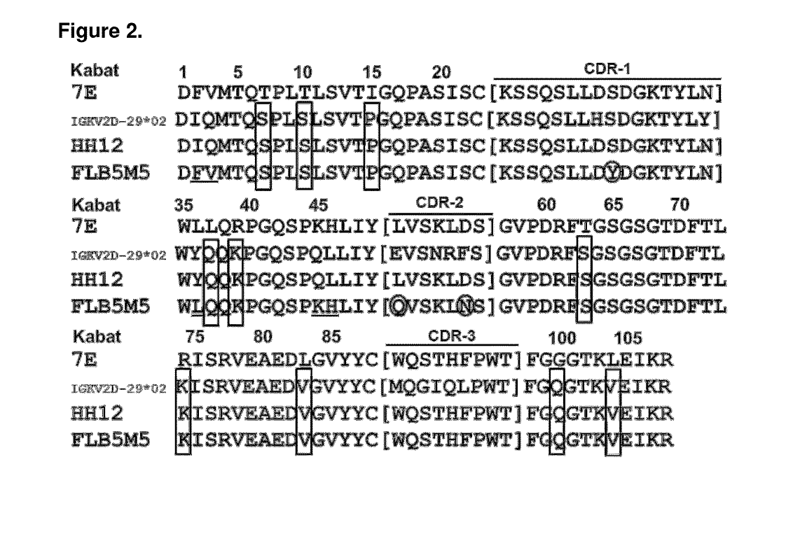

The CDRs were optimized using affinity maturation. Multiple methods of introducing mutations in the CDRs are known in the art, including radiation, chemical mutagens, and error-prone PCR. Three mutations in the CDRs were determined to be beneficial using the affinity maturation method LTM, with one mutation (from serine to tyrosine) found to be especially beneficial. FIG. 2 provides a comparison of the amino acids of 7E, human homolog, HH12, and FLB5M5, in the V_{L} region. In FIG. 2, the amino acids that differ between FLB5M5 and HH12 in the CDRs are enclosed in circles. We surprisingly found that a tyrosine amino acid at position 27E was especially beneficial for binding affinity to IL-20, as is demonstrated in Table 1.

TABLE 1
Comparison of binding kinetics for various specimens with mutations in the CDRs

| | CDRH3 | | CDRL1 | CDRL2 | CDRL3 | | | |
|---|---|---|---|---|---|---|---|---|
| Specimen | 96 | 98 | 27E | 50 | 93 | Kₐ | K_{d} | K_{D} (M) |
| HH12 | S | R | S | L | H | 1.34E+6 | 3.58E−3 | 2.68E−9 |
| G3L | S | R | Y | L | H | 8.12E+5 | 1.07E−3 | 1.31E−9 |
| FLB67 | S | Q | Y | Q | H | 8.88E+5 | 7.13E−4 | 8.03E−10 |
| FLB35 | D | R | Y | L | L | 9.88E+5 | 4.31E−4 | 4.37E−10 |
| FLB35(S) | D | R | S | L | L | 7.03E+5 | 2.83E−3 | 4.03E−9 |

Back mutation was used to identify five amino acids within the light-chain framework region of HH12 to increase the binding affinity to IL-20. In FIG. 2, the amino acids that differ between FLB5M5 and HH12 in the framework are underlined.

Finally, a combination of three mutations in the CDRs and five mutations within the light-chain framework region were all expressed as one humanized anti-IL-20 antibody, FLB5M5.

### EXAMPLES

Selection of Human V Region Framework:

Human germ-line V_{L} and V_{H} sequences with the highest degree of homology with the 7E framework regions were identified from the IMGT data base (http://www.imgt.org/). Amino acid sequences of VH3 and Vκ2 were selected for the V_{H} and V_{L} frameworks, respectively.

HH12 scFv Construction and Display:

HH12 consisted of a complete human framework (VL κ subgroup II and VH subgroup III) with the six complete murine CDR sequences. The HH12 scFv construct was assembled by overlap PCR. An equimolar mixture of oligonucleotides (final 0.4 μM) was PCR-assembled using 0.5 μl of Pfx50 DNA polymerase and 5 μl of Pfx50 buffer (Invitrogen). A second PCR step, including oligonucleotide primers to incorporate 5′ Sfil and 3′ Notl restriction sites, was used for directional subcloning into modified phage display vector pCANTAB5e (Amersham Pharmacia Biotech).

Back Mutation:

The plasmid pTCAE8.3 was used for subcloning and expression of full length antibody in free style 293 cells. The plasmid contains a DNA fragment encoding human κ C_{L} region and human γ C_{H} region. Individual oligonucleotides were synthesized to encode mutations and provide sufficient overlap for PCR priming from the HH12 template. Next, these PCR products were gel-purified, and equimolar aliquots were combined for megaprime PCR to regenerate full-length V_{L}. The V_{L} fragment was subcloned into pTCAE8.3 vector.

LTM Library Construction:

Individual oligonucleotides were synthesized to encode each amino acid substitution for each CDR position and provide sufficient overlap for PCR priming from the HH12 template. PCRs containing LTM oligonucleotide mixtures corresponding to individual CDRs were used to amplify LTM-substituted CDR fragments. Next, these PCR products were gel-purified, and equimolar aliquots were combined for megaprime PCR to regenerate full-length scFv. These scFv fragments were inserted into the pCANTAB5e vector. The ligated DNA was electroporated into E. coli TG1 cells.

Preparation of Phage and Selection of Phage Antibody Libraries

The library stock was grown in log phase, rescued with M13KO7 helper phage (NEB), and amplified overnight in 2YTAK (2YT containing 100 μg/mL ampicillin and 25 μg/mL kanamycin) at 30° C. The phage was precipitated with PEG/NaCl (20% PEG 8000/2.5M NaCl), and then resuspended in PBS. The library was selected using biotinylated IL-20 and streptavidin-coated paramagnetic beads M280 (Dynal). For selection of the LTM library, IL-20 concentrations of 4.0×10⁻⁸ M, 1.0×10⁻⁹ M, 1.0×10⁻¹¹ M, 1.0×10⁻¹² M, and 1.0×10⁻¹³ M were used for selection rounds 1, 2, 3, 4, and 5, respectively. The mixture of phage and antigen was gently rotated for one hour at room temperature, and phage bound to biotinylated antigen was captured using 50˜100 μl of streptavidin-coated M280 magnetic beads for five minutes. After capture of phage, the beads were washed a total of ten times (4×PBST (PBS containing 0.05% Tween 20), 2×PBSM (containing 2% skimmed milk powder), 4×PBS) using a Dynal magnetic particle concentrator. The third, fourth, and fifth washes were performed in competition with 1.4 μM IL-20. Bound phages were eluted from the beads by sequential incubation with 1 ml of 100 mM triethylamine (TEA) for 30 minutes. Eluents were combined and neutralized with 0.5 ml of 1 M Tris HCl (pH 7.4) and half of the eluent was used to infect log phase E. coli TG1.

Expression and Affinity Measurements of HH12 Variants

The genes encoding the V_{H} and V_{L} chains of HH12 and its mutants were inserted into the pTCAE8.3 expression vector. Free style 293 cells were transfected with the construct. After the full-length antibody was purified from the pooled supernatants, competition ELISA and BIACore assays were used to detect the candidate epitope specificity and binding affinity.

Combinatorial Beneficial Clone

Three beneficial HH12 CDR mutations obtained through the LTM screen and five beneficial HH12 framework mutations obtained from back mutation prediction were used to construct combinatorially the FLB5M5 beneficial clone. The protocol was identical to back mutation described above.

IL-20 Binding Activity

The human IL-20-binding kinetics of each purified anti-IL-20 antibody was estimated by surface plasmon resonance measurements using the BIAcore T100 biosensor system. The anti-IL-20 Ab was captured on an anti-human IgG immobilized CM5 sensor chip. The immobilized level of anti-human IgG was about 9,000-10,000 RU and the capture level of anti-IL-20 antibody was about 350-400 RU. Binding was carried out at constant flow rates of 30 μL/min of IL-20 at various dilutions in HEPES buffered saline (BIA certified) for 60 seconds. Dissociations were carried out by passing through HEPES buffer for 480 seconds. Regeneration of the surface was carried out by infecting 10 mM Glycine pH 2.0/1.5 (50:50) for 40 seconds. The IL-20 affinity of each of the anti-IL-20 antibodies were calculated from an affinity binding curve fit using the predefined model (1:1 binding) provided by Biacore T100 evaluation software 2.0. The binding affinity data are summarized in Table 2.

TABLE 2
Comparison of binding affinities
to IL-20 of 7E, HH12, and FLB5M5

| Specimen | kₐ | k_{off} | K_{D} (M) |
|---|---|---|---|
| 7E | 9.11E+5 | 7.11E−4 | 7.81E−10 |
| HH12 | 1.34E+6 | 3.58E−3 | 2.68E−9 |
| FLB5M5 | 1.33E+6 | 3.82E−4 | 2.88E−10 |

IL20R2/IL20R2 BaF3 Proliferation Assay

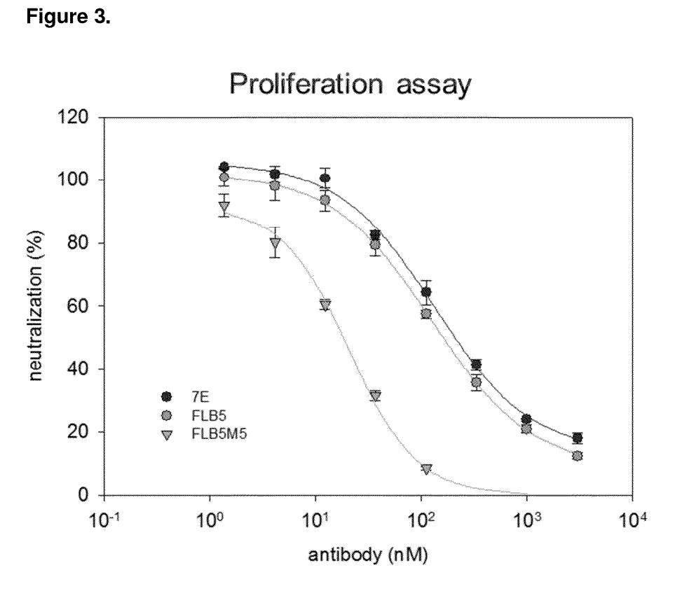

The biological activity of the expressed human recombinant IL-20 was measured in a proliferation assay employing BaF3 cells stably transfected with full-length human IL-20 receptor complexes IL-22R and IL-20R2 as the targets. BaF3 cells are murine precursor B cells of the early lymphoblastoid cell lineage dependent on IL-3 for viability and proliferation. The cells were cultured in RPMI medium containing 10% fetal bovine serum and 1 ng/mL IL-3. In the proliferation assay, BaF-3(IL-20R2/IL22R) cells were seeded in microtitre wells at 10⁴ cells per well in medium without IL-3 for 2 h at 37° C. in a 5% CO₂ incubator. Then, BaF-3(IL-20R2/IL22R) cells were cultured with pre-incubated 300 pM human cytokine IL20 and an increasing amount of antibody (three-fold dilutions from 1000 to 0.15 nM) for another 72 h. AlamarBlue (Promega) was used as a colorimetric growth indicator and was added to the cultures in the last 6 h of stimulation. The microtitre plates were read on a fluorometer at 530 nm excitation and 580 nm emission. The fluorescent readout was analyzed using SigmaPlot Software to find the half maximal response (EC₅₀) for the anti-human IL20 antibody. FIG. 3 is a graphical representation of the results from the proliferation assay. As summarized in Table 3, FLB5M5 exhibited a better neutralization activity than 7E toward IL-20.

TABLE 3
Comparison of neutralizing activity
toward IL-20 of 7E and FLB5M5

| Specimen | EC₅₀ (nM) |
|---|---|
| 7E | 134.63 |
| FLB5M5 | 20.76 |

Collagen-Induced Arthritis Rat Model:

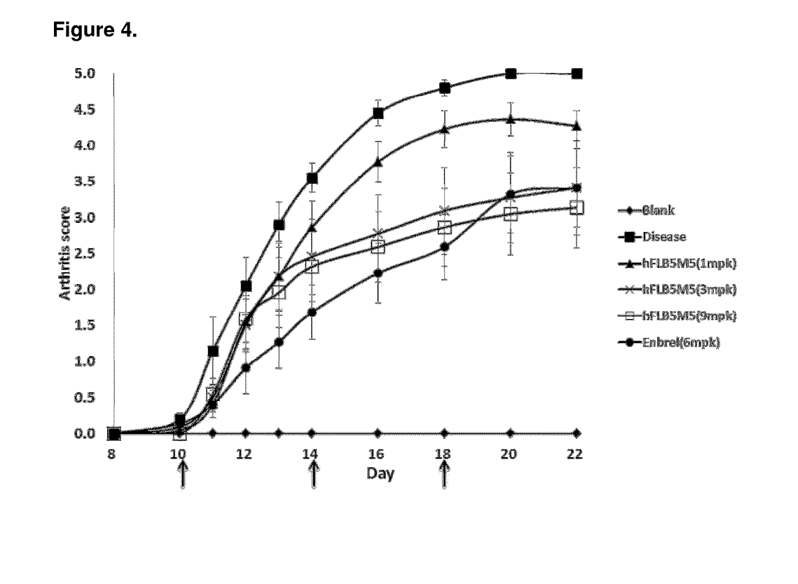

Six-week-old male SD rats were immunized with type II collagen on day 0 and day 7. After the onset of arthritis, which typically occurs on day 10-13, the treatments were started. Test articles were administrated twice per week for a total of three injections. Animals of all treatment groups received a single bolus subcutaneous injection over the back on days 10, 14, and 18. Measurements were repeated at 1 mg/kg, 3 mg/kg, and 9 mg/kg. The body weight, hind-paws thickness (PTH), and arthritic score (AS) were assessed to evaluate the therapeutic efficacy at three doses. FIG. 4 provides the AS profiles of healthy, diseased, and treated rats. In FIG. 4, arrows indicate the administration of FLB5M5. As is evident, FLB5M5 effectively reduced the arthritis score of the diseased rats. It is expected that similar effects would be observed when treating other inflammatory diseases.

Thus, a humanized monoclonal antibody with three mutated amino acids in the CDRs, relative to its parental mouse anti-IL-20 monoclonal antibody 7E, and five mutated amino acids of the light-chain framework region, relative to the amino acids of the light-chain framework region of human Vκ2 is disclosed. Also disclosed is a specific mutation in the V_{L} CDR-1 believed to play an important role in binding affinity to IL-20. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A humanized antibody derived from mouse monoclonal antibody 7E.

2. The humanized antibody of claim 1, comprising three complementarity determining regions (CDRs), wherein three amino acids in the CDRs are different from the amino acids of the CDRs of mouse monoclonal antibody 7E.

3. The humanized antibody of claim 1, comprising three CDRs and a light-chain framework region, wherein five amino acids in the light-chain framework region are different from the amino acids of the light-chain framework region of human VκK2.

4. The humanized antibody of claim 3, comprising three CDRs and a light-chain framework region, wherein three amino acids in the CDRs are different from the amino acids of the CDRs of mouse monoclonal antibody 7E.

5. The humanized antibody of claim 4 which is optimized to interact with interleukin-20 (IL-20).

6. The humanized antibody of claim 4 with a binding affinity to IL-20 better than the binding affinity of mouse monoclonal antibody 7E to IL-20.

7. The humanized antibody of claim 4 with a neutralizing activity toward IL-20 better than the neutralizing activity of mouse monoclonal antibody 7E toward IL-20.

8. The humanized antibody of claim 2, wherein at least one of the three amino acids in the CDRs is Tyrosine.

9. The humanized antibody of claim 4, wherein at least one of the three amino acids in the CDRs is Tyrosine.

10. A process for the production of the humanized antibody of claim 5, said process comprising:
a) sequencing mouse monoclonal antibody 7E;
b) selecting a framework donor antibody;
c) replacing CDRs of the framework donor antibody with CDRs from the mouse monoclonal antibody 7E;
d) replacing five amino acids from the framework donor antibody light-chain framework region with amino acids from the mouse monoclonal antibody 7E; and
e) mutating three amino acids from the CDRs.

11. A method of treating an inflammatory disease in a mammal, said method comprising administering the humanized antibody of claim 4 in an amount sufficient to treat said inflammatory disease.

12. The method of claim 11 wherein the mammal is a mouse.

13. The method of claim 11 wherein the mammal is a human.

14. The method of claim 11 wherein the inflammatory disease is rheumatoid arthritis.

15. The method of claim 11 wherein the inflammatory disease is psoriasis.

16. The method of claim 11 wherein the inflammatory disease is atherosclerosis.

17. The method of claim 11 wherein the inflammatory disease is stroke.

18. The method of claim 11 wherein the inflammatory disease is osteoporosis.
